(19) United States
(12) Patent Application Publication
(10) Pub. No.: US 2024/0186002 A1
(43) Pub. Date: Jun. 6, 2024

(54) INFORMATION PROCESSING APPARATUS, FEATURE QUANTITY EXTRACTION METHOD, TRAINING DATA GENERATION METHOD, ESTIMATION MODEL GENERATION METHOD, STRESS LEVEL ESTIMATION METHOD, AND STORAGE MEDIUM

(71) Applicant: NEC Corporation, Minato-ku, Tokyo (JP)

(72) Inventors: Yoshiki NAKASHIMA, Tokyo (JP); Masanori TSUJIKAWA, Tokyo (JP)

(73) Assignee: NEC Corporation, Minato-ku, Tokyo (JP)

(21) Appl. No.: 18/285,433

(22) PCT Filed: Apr. 8, 2021
(86) PCT No.: PCT/JP2021/014959

§ 371 Date: Oct. 3, 2023

(51) Int. Cl. G16H 50/20; G16H 50/30

(52) U.S. Cl. CPC G16H 50/20; G16H 50/30

(57) ABSTRACT

In order to appropriately extract feature quantities for use in machine learning or estimation of a stress level, an information processing apparatus (1, 4) includes: an identification means (11, 404) of identifying, as a time zone of interest, a time zone in which a chronic stress tendency is notably shown in biological signals which have been acquired from a subject over a predetermined time period; and an extraction means (12, 405) of extracting one or more feature quantities from biological signals acquired in the time zone of interest which has been identified, the one or more feature quantities being used in machine learning of an estimation model for estimating a stress level or used in estimation of a stress level using the estimation model.

### TECHNICAL FIELD

The present invention relates to a technique for extracting a feature quantity which is used in machine learning of a stress level estimation model or in estimation of a stress level using the estimation model.

### BACKGROUND ART

In recent years, there are increasing cases where employees suffer from mental problems such as depression due to occupational stress, resulting in quitting jobs or taking leaves of absence. Along with the circumstances, there is also a problem of increasing burdens on companies that maintain and secure employees. Against this background, studies on stress monitoring are underway. For example, studies are also underway on a technique for generating a stress level estimation model using measurement data such as body motion data and biological data of a subject, and estimating a stress level of the subject using the generated estimation model.

For example, Patent Literature 1 discloses a stress estimation apparatus and a stress estimation method using biological signals.

### CITATION LIST

### Patent Literature

[Patent Literature 1]
PCT International Publication No. WO2019/159252A1

### SUMMARY OF INVENTION

### Technical Problem

For the stress estimation apparatus, it is demanded to further improve accuracy of stress estimation. Obtaining appropriate feature quantities for use in machine learning and estimation of a stress level leads to improvement in accuracy of stress estimation.

An example aspect of the present invention is accomplished in view of the above problems, and its example object is to provide a technique for extracting an adequate feature quantity which is used in machine learning of a stress level estimation model or in estimation of a stress level using the estimation model.

### Solution to Problem

An information processing apparatus in accordance with an example aspect of the present invention includes: an identification means of identifying, as a time zone of interest, a time zone in which a chronic stress tendency is notably shown in biological signals which have been acquired from a subject over a predetermined time period; and an extraction means of extracting one or more feature quantities from biological signals acquired in the time zone of interest which has been identified, the one or more feature quantities being used in machine learning of an estimation model for estimating a stress level or used in estimation of a stress level using the estimation model.

A feature quantity extraction method in accordance with an example aspect of the present invention includes: identifying, as a time zone of interest by at least one processor, a time zone in which a chronic stress tendency is notably shown in biological signals which have been acquired from a subject over a predetermined time period; and extracting, by the at least one processor, one or more feature quantities from biological signals acquired in the time zone of interest which has been identified, the one or more feature quantities being used in machine learning of an estimation model for estimating a stress level or used in estimation of a stress level using the estimation model.

A feature quantity extraction program in accordance with an example aspect of the present invention causes a computer to carry out: an identification process of identifying, as a time zone of interest, a time zone in which a chronic stress tendency is notably shown in biological signals which have been acquired from a subject over a predetermined time period; and an extraction process of extracting one or more feature quantities from biological signals acquired in the time zone of interest which has been identified, the one or more feature quantities being used in machine learning of an estimation model for estimating a stress level or used in estimation of a stress level using the estimation model.

### Advantageous Effects of Invention

According to an example aspect of the present invention, it is possible to extract an adequate feature quantity which is used in machine learning of a stress level estimation model or in estimation of a stress level using the estimation model.

### BRIEF DESCRIPTION OF DRAWINGS

FIG. 1 is a block diagram illustrating a configuration of an information processing apparatus in accordance with a first example embodiment of the present invention.

FIG. 2 is a flowchart illustrating a flow of a feature quantity extraction method in accordance with the first example embodiment of the present invention.

FIG. 3 is a block diagram illustrating a configuration of an information processing apparatus in accordance with second through fifth example embodiments of the present invention.

FIG. 4 is a flowchart illustrating a flow of an information processing method in a training phase in accordance with second and third example embodiments of the present invention.

FIG. 5 is a flowchart illustrating a flow of an information processing method in an inference phase in accordance with second and third example embodiments of the present invention.

FIG. 6 is a flowchart illustrating a flow of an information processing method in a training phase in accordance with the fourth example embodiment of the present invention.

FIG. 7 is a flowchart illustrating a flow of an information processing method in an inference phase in accordance with the fourth example embodiment of the present invention.

FIG. 8 is a flowchart illustrating a flow of an information processing method in a training phase in accordance with the fifth example embodiment of the present invention.

FIG. 9 is a flowchart illustrating a flow of an information processing method in an inference phase in accordance with the fifth example embodiment of the present invention.

FIG. 10 is a block diagram illustrating an example of a hardware configuration of the information processing apparatus according to each of the example embodiments of the present invention.

### EXAMPLE EMBODIMENTS

### First Example Embodiment

The inventors of the present invention have conceived that accuracy of stress estimation is improved by selectively narrowing down biological signals used for creation of an estimation model or for stress estimation, among biological signals which have been acquired from a subject over a predetermined time period, and have completed the present invention. Specifically, the inventors of the present invention have conceived of paying attention to biological signals in a time zone in which a chronic stress tendency is notably shown in the biological signals when a subject is in a particular state, and have completed the present invention. The following description will discuss some example embodiments of the present invention in detail with reference to the drawings.

First, the following description will discuss a first example embodiment of the present invention. The first example embodiment is a basic form of example embodiments described later.

### <Configuration of Information Processing Apparatus>

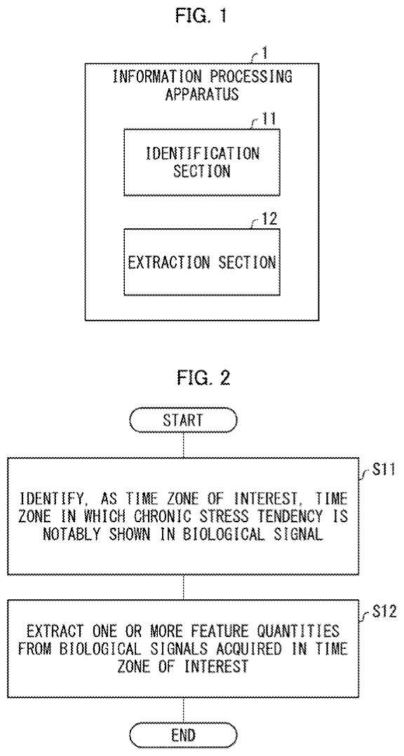

FIG. 1 is a block diagram illustrating a configuration of an information processing apparatus 1. As illustrated in FIG. 1, the information processing apparatus 1 is configured to include an identification section 11 and an extraction section 12. The identification section 11 is configured to realize the identification means in the present example embodiment. The extraction section 12 is configured to realize the extraction means in the present example embodiment.

The identification section 11 identifies, as a time zone of interest, a time zone in which a chronic stress tendency is notably shown in biological signals which have been acquired from a subject over a predetermined time period.

The extraction section 12 extracts one or more feature quantities from biological signals acquired in the time zone of interest which has been identified by the identification section 11, the one or more feature quantities being used in machine learning of an estimation model for estimating a stress level or used in estimation of a stress level using the estimation model.

As described above, the information processing apparatus 1 in accordance with the present example embodiment employs the configuration of including the identification section 11 and the extraction section 12 described above.

According to the above configuration, first, a time zone of interest in which a particular tendency caused due to a chronic stress is notably shown in biological signals is identified. Then, a feature quantity is extracted from a biological signal of the subject which has been acquired in the time zone of interest. Consequently, it is possible to bring about an example advantage of extracting an adequate feature quantity which is used in machine learning of a stress level estimation model or in estimation of a stress level using the estimation model.

Then, the feature quantity that has been obtained by the above configuration and is highly correlated with chronic stress can be used, for example, to generate training data for use in machine learning of an estimation model that estimates a chronic stress level, together with correct answer data indicating a chronic stress level corresponding to the feature quantity. Consequently, it is possible to efficiently construct an estimation model that achieves high estimation accuracy of chronic stress.

In another example, the feature quantity that has been obtained by the above configuration and is highly correlated with chronic stress can be used as an input value for the above estimation model. Consequently, it is possible to carry out estimation of chronic stress with higher accuracy.

The information processing apparatus 1 can be realized by a computer and a program of the computer. The program is a feature quantity extraction program that causes the computer to function as the identification section 11 and the extraction section 12 described above. According to the feature quantity extraction program, it is possible to obtain an example advantage similar to that of the foregoing information processing apparatus 1.

### <Flow of Feature Quantity Extraction Method>

FIG. 2 is a flowchart illustrating a flow of a feature quantity extraction method which is carried out by the information processing apparatus 1.

In step S11, the identification section 11 identifies, as a time zone of interest, a time zone in which a chronic stress tendency is notably shown in biological signals which have been acquired from a subject over a predetermined time period.

In step S12, the extraction section 12 extracts one or more feature quantities from biological signals acquired in the time zone of interest which has been identified by the identification section 11, the one or more feature quantities being used in machine learning of an estimation model for estimating a stress level or used in estimation of a stress level using the estimation model.

As described above, the feature quantity extraction method according to the present example embodiment employs the configuration of including steps S11 and S12.

Therefore, according to the feature quantity extraction method in accordance with the present example embodiment, it is possible to bring about an example advantage of extracting an adequate feature quantity which is used in machine learning of a stress level estimation model or in estimation of a stress level using the estimation model, as with the foregoing information processing apparatus 1.

### <Variation>

The extraction section 12 in accordance with the present example embodiment may extract feature quantities using a biological signal acquired in the time zone of interest described above and a biological signal acquired in another predetermined time zone based on the time zone of interest.

The identification section 11 may identify, as a time zone of interest, a time zone in which biological signals show a notable behavior in one day, and the extraction section 12 may extract a feature quantity based on a change in the biological signal at a start time or an end time of the identified time zone of interest.

In step S11 described above, the identification section 11 may identify, as a time zone of interest, a time zone in which biological signals show a notable behavior in one day. In step S12 described above, the extraction section 12 may further extract a feature quantity based on a change in a biological signal at a start time or an end time of the time zone of interest.

According to the above configuration and method, first, a feature quantity is extracted from a biological signal which has been acquired at least in a time zone in which biological signals tend to show a notable behavior in one day, as a time zone in which a chronic stress tendency is notable. Next, the extraction section 12 further extracts a feature quantity based on a change in the biological signals which are observed before and after the time zone of interest, in addition to the foregoing feature quantity.

In the time zone of interest, a particular tendency caused due to chronic stress is notably shown in biological signals. Therefore, even in a change in a biological signal at a start time or an end time of the time zone of interest, a particular tendency caused due to chronic stress is notably shown. Therefore, extracting a feature quantity based on a change in a biological signal at a start time or an end time of the time zone of interest leads to an increase in estimation accuracy of chronic stress.

For example, the extraction section 12 may extract, as a feature quantity, an amount of change in a predetermined index value of a biological signal which is observed at a start time or an end time of the time zone of interest. Here, the predetermined index value of a biological signal can be the biological signal itself (unprocessed data, so-called raw data output from a sensor), or may alternatively be a calculated value which has been calculated based on the biological signal.

In a case where it is known in advance that an amount of change in the predetermined index values of biological signals before and after the time zone of interest is significantly correlated with chronic stress, extraction of the amount of change as a feature quantity leads to an increase in estimation accuracy of chronic stress.

Note that the above “time zone in which biological signals show a notable behavior” may be a specific time zone in which the predetermined index value of a biological signal indicates a relatively high value in one day. The predetermined index value is, for example, a value indicating: an index obtained from time series data of biological signals; an index obtained from frequency data of biological signals; another important index for predicting chronic stress; or the like. For example, the index value can be a value representing a heart rate, an amount of perspiration, a respiration rate, a pulse wave, a body temperature, or the like which can be derived from biological signals.

For example, the identification section 11 may identify, as a time zone of interest, a time zone in which a heart rate tends to be high in one day. According to the above configuration, a feature quantity is extracted from a biological signal which has been acquired in a particular time zone in which a heart rate tends to be high in one day, as the time zone in which a chronic stress tendency is notable. Consequently, it is possible to bring about an example advantage of extracting an adequate feature quantity which is used in machine learning of a stress level estimation model or in estimation of a stress level using the estimation model. Moreover, it is possible to efficiently construct an estimation model that achieves high estimation accuracy of chronic stress, and to carry out estimation of chronic stress with higher accuracy.

### Second Example Embodiment

The following description will discuss a second example embodiment of the present invention in detail with reference to the drawings. The same reference numerals are given to constituent elements which have functions identical with those described in the first example embodiment, and descriptions as to such constituent elements are not repeated.

The inventors of the present invention have conceived that a chronic stress tendency is notably shown in biological signals in a time zone in which a predetermined index value of a biological signal (e.g., a heart rate, an amount of perspiration, or the like) is relatively high. Furthermore, it is known that a predetermined index value of a biological signal reaches, based on a circadian rhythm, a peak at around a time in the morning (e.g., 10:00 a.m.) and tends to be high in predetermined time zones before and after that time in the morning.

Therefore, in the present example embodiment, for example, as a time zone of interest in which a chronic stress tendency is notably shown in biological signals, predetermined time zones before and after a time in the morning at which a predetermined index value of a biological signal reaches a peak based on a circadian rhythm are identified.

### <Configuration of Information Processing Apparatus>

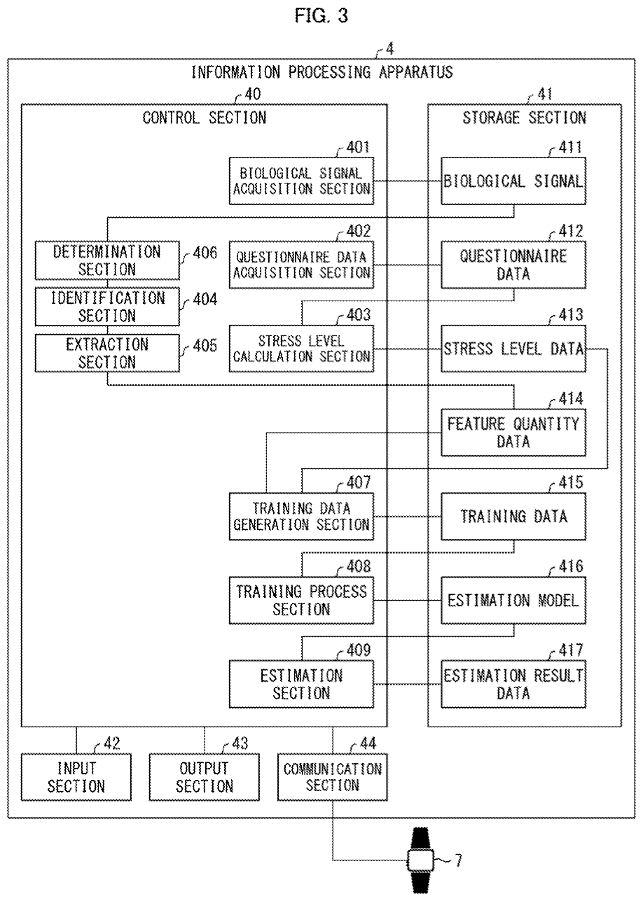

FIG. 3 is a block diagram illustrating a configuration of an information processing apparatus 4. FIG. 3 also illustrates a wearable terminal 7 as an example of an apparatus that measures a biological signal.

The wearable terminal 7 measures a state of a wearer and outputs a biological signal as an output value. For example, the wearable terminal 7 has a function of detecting a heart rate of the wearer and a function of detecting perspiration of the wearer. When the subject wears the wearable terminal 7, heart rate data indicating a heart rate of the subject and perspiration data indicating an amount of perspiration of the subject are generated as biological signals. These biological signals are transmitted to the information processing apparatus 4.

The wearable terminal 7 may further include a triaxial acceleration sensor. An output value of the acceleration sensor may be transmitted from the wearable terminal 7 to the information processing apparatus 4 as a biological signal. When the subject wears the wearable terminal 7, body motion of the subject is detected by the acceleration sensor. Since it is known that body motion is correlated with the stress level of the subject, it is possible to estimate a stress level by using an output value of the acceleration sensor as a biological signal. Note that the acceleration sensor is not limited to a triaxial acceleration sensor, and may be a uniaxial or biaxial acceleration sensor. Moreover, physical activity of the subject can be ascertained from an output value of the wearable terminal 7 as an acceleration sensor. Therefore, it is also possible to determine whether or not a biological signal being measured is derived from physical activity.

In the following descriptions, an example is described in which, in order to simplify the descriptions, a single wearable terminal 7 measures all biological signals acquired for a subject and transmits the signals to the information processing apparatus 4. Note, however, that the information processing apparatus 4 may acquire several kinds of biological signals from different apparatuses, respectively.

The information processing apparatus 4 includes a control section 40 that comprehensively controls components of the information processing apparatus 4, and a storage section 41 that stores various kinds of data used by the information processing apparatus 4. The information processing apparatus 4 further includes: an input section 42 that receives input of data with respect to the information processing apparatus 4; an output section 43 for outputting data from the information processing apparatus 4; and a communication section 44 for carrying out communication between the information processing apparatus 4 and another apparatus (e.g., the wearable terminal 7).

The control section 40 includes a biological signal acquisition section 401, a questionnaire data acquisition section 402, a stress level calculation section 403, an identification section 404, an extraction section 405, a determination section 406, a training data generation section 407, a training process section 408, and an estimation section 409. The storage section 41 stores a biological signal 411, questionnaire data 412, stress level data 413, feature quantity data 414, training data 415, an estimation model 416, and estimation result data 417.

The identification section 404 is configured to realize the identification means in the present example embodiment. The extraction section 405 is configured to realize the extraction means in the present example embodiment. The determination section 406 is configured to realize the determination means in the present example embodiment. The estimation section 409 is configured to realize the estimation means. Note that, in the present example embodiment, the determination section 406 can be omitted. The determination section 406 will be described in an example embodiment described later.

The biological signal acquisition section 401 acquires a biological signal of a subject and causes the storage section 41 to store the acquired biological signal. The biological signal stored in the storage section 41 is a biological signal 411. The biological signal 411 can include a signal used for generation of training data 415 and a signal used for estimation of a stress level.

The questionnaire data acquisition section 402 acquires a result of a questionnaire pertaining to a stress level of the subject in a time period in which biological signals 411 that are used for generation of training data 415 have been measured, and causes the storage section 41 to store questionnaire data 412 indicating the acquired result. This questionnaire is a questionnaire answered by the subject in order to calculate the stress level of the subject.

The questionnaire only needs to have content that reflects a stress level of a subject, and may be a stress questionnaire of, for example, perceived stress scale (PSS). The stress questionnaire of PSS is a questionnaire in the form in which a subject selects an applicable one from a plurality of options, for each of a plurality of questions regarding how the subject feels and behaves during a time period in question.

The stress level calculation section 403 calculates a stress level of a subject using the questionnaire data 412, and causes the storage section 41 to store stress level data 413 that indicates the calculated stress level. Any method for calculating the stress level can be applied. For example, in a case where the questionnaire data 412 is data indicating a result of a stress questionnaire of PSS, the stress level calculation section 403 calculates a PSS score.

The identification section 404 identifies, as a time zone of interest, a time zone in which a chronic stress tendency is notably shown in biological signals which have been acquired from a subject over a predetermined time period.

In the present example embodiment, the identification section 404 identifies, as the time zone of interest, predetermined time zones before and after a time in the morning at which a predetermined index value of a biological signal reaches a peak based on a circadian rhythm.

For example, the predetermined index value can be a heart rate. The inventors of the present invention have conceived that a chronic stress tendency is notably shown in biological signals in a time zone in which a heart rate is relatively high. Furthermore, it is known that a heart rate reaches, based on a circadian rhythm, a peak at around a time in the morning (e.g., 10:00 a.m.) and tends to be high in predetermined time zones before and after that time in the morning. Therefore, the identification section 404 may be configured to identify, as the time zone of interest, predetermined time zones (e.g., a time zone between around 7:00 a.m. and around 13:00) before and after 10:00 at which the heart rate indicated by the heart rate data reaches a peak based on a circadian rhythm, for example.

In a case where a peak time in the circadian rhythm of a biological signal (such as an amount of perspiration) different from the heart rate is known in advance, the identification section 404 may identify, as a time zone of interest, predetermined time zones in which the amount of perspiration is relatively large before and after the time at which the amount of perspiration indicated by the perspiration data reaches the peak.

In another example, the identification section 404 may analyze biological signals which have been acquired over a predetermined time period, and identify a time point S at which an index contributing to estimation of a chronic stress tendency has started to notably appear in biological signals, and a time point E at which the index contributing to estimation of the notable chronic stress tendency has ended. Then, the identification section 404 may identify, as the time zone of interest, a time zone from the time point S to the time point E which have been identified. The index that contributes to estimation of a chronic stress tendency in biological signals can be, for example, a heart rate. For example, the identification section 404 may identify, as a time zone of interest, a time zone from (i) a time point S at which a value (such as a numerical value itself of a heart rate, which is an example of the above index, or an index value related to a chronic stress tendency which has been calculated from a heart rate) reaches a predetermined threshold to (ii) a time point E at which the value falls below the predetermined threshold.

The extraction section 405 extracts one or more feature quantities from biological signals acquired in the time zone of interest which has been identified, the one or more feature quantities being used in machine learning of an estimation model for estimating a stress level or used in estimation of a stress level using the estimation model. For example, the extraction section 405 may calculate a feature quantity from the biological signal 411 and cause the storage section 41 to store the calculated feature quantity. The feature quantity data 414 is data which indicates a feature quantity extracted by the extraction section 405 and stored in the storage section 41. The feature quantity data 414 can include a feature quantity used for generation of training data 415. Hereinafter, a feature quantity used for generation of training data 415 is referred to as a training feature quantity. That is, the training feature quantity is a feature quantity used for machine learning of a stress level estimation model.

The feature quantity data 414 can also include at least one or more feature quantities extracted from biological signals in the time zone of interest. That is, the feature quantity data 414 may include a plurality of types of feature quantities. The feature quantity data 414 can include a feature quantity which has been extracted by taking into account a biological signal falling outside the time zone of interest. For example, the feature quantity data 414 may include a feature quantity which has been extracted based on a change in a biological signal at a start time or an end time of a time zone of interest.

Furthermore, the feature quantity data 414 can also include a feature quantity used for estimation of a stress level. Hereinafter, the feature quantity used for estimation of a stress level is referred to as an estimating feature quantity. The estimating feature quantity is a feature quantity that has been generated from a biological signal obtained, in a predetermined time period for which a stress level is to be measured, from a subject whose stress level is to be estimated.

The training data generation section 407 generates training data by associating, as correct answer data, a stress level indicated in the stress level data 413 with a combination of one or more training feature quantities which have been extracted by the extraction section 405. Then, the training data generation section 407 causes the storage section 41 to store the generated training data as training data 415.

The training process section 408 generates, by training using the training data 415, an estimation model for which the one or more training feature quantities extracted by the extraction section 405 are used as explanatory variables and from which the stress level is obtained as an objective variable. Then, the training process section 408 causes the storage section 41 to store the generated estimation model as an estimation model 416.

The estimation section 409 estimates the stress level of the subject using an estimating feature quantity that has been generated from a biological signal of the subject. More specifically, the estimation section 409 calculates an estimation value of the stress level by inputting, into the estimation model 416, an estimating feature quantity included in the feature quantity data 414. Then the estimation section 409 causes the storage section 41 to store estimation result data 417 indicating a stress level estimation result.

### <Variation>

Influence of chronic stress on biological signals may differ between a male and a female. For example, there is a paper reporting that, in regard to heart rate data based on a circadian rhythm, there is a tendency for females that the heart rate decreases under chronic stress, whereas there is a tendency for males that the heart rate increases under chronic stress.

In view of this, it is preferable that estimation models 416 are separately generated according to gender. Specifically, the extraction section 405 extracts a feature quantity of a male from a biological signal of the male which has been measured in a time zone of interest (e.g., a time zone between around 7:00 a.m. and around 13:00) in order to generate an estimation model for males. Moreover, the extraction section 405 extracts a feature quantity of a female from a biological signal of the female which has been measured in the time zone of interest in order to generate an estimation model for females. The training data generation section 407 generates training data for males for generating an estimation model for males using feature quantities of males, and generates training data for females for generating an estimation model for females using feature quantities of females. Thus, the training process section 408 can separately generate, according to gender, estimation models 416 using the pieces of training data that are separated between males and females. Then, the estimation section 409 can estimate a stress level for a male subject using the estimation model for males, and estimate a stress level for a female subject using the estimation model for females.

The foregoing components included in the information processing apparatus 4 can be realized by a plurality of computers. For example, a feature quantity extraction apparatus can be realized which includes: a control section including a biological signal acquisition section 401, an identification section 404, an extraction section 405, and a determination section 406; and a storage section 41 that stores a biological signal 411 and feature quantity data 414. A training data generation apparatus can be realized which includes: a control section 40 including a questionnaire data acquisition section 402, a stress level calculation section 403, and a training data generation section 407; and a storage section 41 that stores questionnaire data 412, stress level data 413, and training data 415. An estimation model generation apparatus can be realized which includes: a control section 40 including a training process section 408; and a storage section 41 that stores an estimation model 416. An estimation apparatus can be realized which includes: a control section 40 including an estimation section 409; and a storage section 41 that stores estimation result data 417. The estimation apparatus can be configured to include constituent elements of the feature quantity extraction apparatus, specifically, the identification section 404 and the extraction section 405. Moreover, an information processing system that is configured such that a wearable terminal 7, a feature quantity extraction apparatus, a training data generation apparatus, an estimation model generation apparatus, and an estimation apparatus are communicably connected to each other via a communication network is also encompassed in the scope of the present invention.

### <Flow of Information Processing Method in Training Phase>

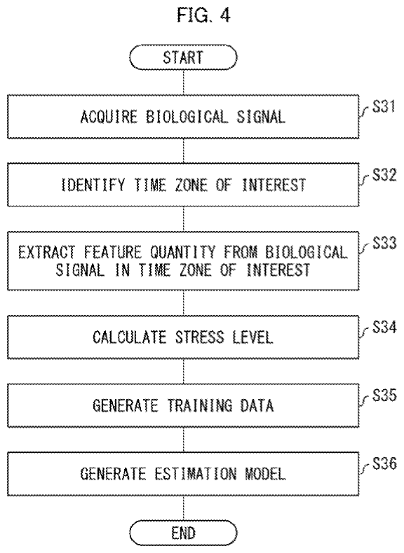

FIG. 4 is a flowchart illustrating a flow of an information processing method in a training phase, which is carried out by the information processing apparatus 4 in accordance with the second example embodiment of the present invention. The information processing method illustrated in FIG. 4 includes, for example, the feature quantity extraction method, the training data generation method, and the estimation model generation method in accordance with example aspects of the present invention. In the present example embodiment, steps S32 and S33 are processes for realizing the feature quantity extraction method, step S35 is a process for realizing the training data generation method, and step S35 is a process for realizing the estimation model generation method.

The processes describe above can also be realized by a program. That is, a feature quantity extraction program that causes a computer to carry out the processes of steps S32 and S33 is also encompassed in the scope of the present example embodiment. Similarly, a training data generation program that causes a computer to carry out the process in step S35 of generating training data using the feature quantity extracted in step S33 is also encompassed in the scope of the present example embodiment. Moreover, an estimation model generation program that causes a computer to carry out the process in step S36 of generating an estimation model using the training data generated in step S35 is also encompassed in the scope of the present example embodiment.

Note that a series of processes of the information processing method illustrated in FIG. 4 can be carried out by the above information processing system in place of the information processing apparatus 4. In this case, an execution subject of steps S31 through S33 is the foregoing feature quantity extraction apparatus, an execution subject of steps S34 and S35 is the foregoing training data generation apparatus, and an execution subject of step S36 is the foregoing estimation model generation apparatus.

In the following descriptions, an example will be described in which an estimation model is generated using, as biological signals, heart rate data and perspiration data of a subject which have been measured by the wearable terminal 7. The biological signal to be used can be a biological signal of a single subject or can be biological signals of a plurality of subjects. However, it is preferable that the biological signal used is a biological signal of a subject whose response to stress is close to that of a subject whose stress level is to be estimated. Further, in regard to each subject, it is assumed that a questionnaire has been answered for calculating a stress level during a time period in which biological signals have been measured, and a result thereof is stored in the storage section 41 as questionnaire data 412. Further, all feature quantities in FIG. 4 are the above described training feature quantities, and in the description of FIG. 4, each of the feature quantities is simply referred to as a feature quantity.

In step S31, the biological signal acquisition section 401 acquires biological signals used for generation of an estimation model. As described above, biological signals acquired here are heart rate data and perspiration data of the subject which have been measured by the wearable terminal 7. Then, the biological signal acquisition section 401 causes the storage section 41 to store the acquired biological signals as biological signals 411.

In step S32, the identification section 404 identifies, as a time zone of interest, a time zone in which a chronic stress tendency is notably shown in the biological signals among the biological signals 411 which have been stored in step S31. In the present example embodiment, for example, a time zone in which the biological signals show a notable behavior in one day is identified as the time zone of interest. In the present example embodiment, more specifically, predetermined time zones before and after a time in the morning at which a predetermined index value of a biological signal reaches a peak based on a circadian rhythm are identified as the time zone of interest. For example, the identification section 404 may identify, as the time zone of interest, predetermined time zones (e.g., from 7:00 to 13:00) before and after 10:00 at which a heart rate obtained from a biological signal reaches a peak.

In step S33, the extraction section 405 extracts feature quantities from biological signals which have been measured in the time zone of interest which has been identified in step S32, among the biological signals 411 which have been stored in step S31. Specifically, the extraction section 405 may extract a plurality of types of feature quantities from each of the heart rate data and the perspiration data. The extracted feature quantities are stored in the storage section 41 as feature quantity data 414.

In step S34, the stress level calculation section 403 calculates a stress level of the subject using the questionnaire data 412. Then, the stress level calculation section 403 causes the storage section 41 to store the calculated stress level as stress level data 413. Note that the process of step S34 may be carried out before step S31, or may be carried out concurrently with steps S31 through S33, as long as the process of step S34 is carried out before step S35.

In step S35, the training data generation section 407 generates training data by associating, as correct answer data, a stress level which has been calculated in step S34 and which is indicated in the stress level data 413 with a combination of one or more feature quantities which have been extracted in step S33. Then, the training data generation section 407 causes the storage section 41 to store the generated training data as training data 415.

In step S36, the training process section 408 generates a stress level estimation model by machine learning using the training data generated in step S35. Note that step S36 may include a series of processes in which a plurality of estimation models are generated, estimation accuracy of each of the generated estimation models is evaluated, and an ultimate estimation model is selected based on the evaluation results. Then, the training process section 408 causes the storage section 41 to store the generated estimation model as an estimation model 416. Thus, the estimation model generation method ends.

### <Flow of Information Processing Method in Inference Phase>

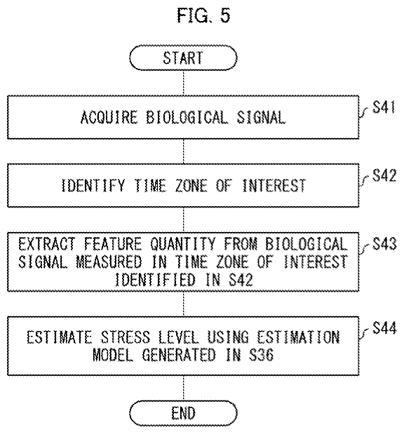

FIG. 5 is a flowchart illustrating a flow of an information processing method in an inference phase, which is carried out by the information processing apparatus 4 in accordance with the second example embodiment of the present invention. The information processing method illustrated in FIG. 5 includes, for example, the feature quantity extraction method and the stress level estimation method in accordance with example aspects of the present invention. In the present example embodiment, steps S42 and S43 are processes for realizing the feature quantity extraction method, and step S44 is a process for realizing the stress level estimation method.

The processes describe above can also be realized by a program. That is, a feature quantity extraction program that causes a computer to carry out the processes of steps S42 and S43 is also encompassed in the scope of the present example embodiment. Moreover, a stress level estimation program that causes a computer to carry out the process in step S44 of estimating a stress level by inputting the feature quantity extracted in step S43 into the estimation model generated in step S36 is also encompassed in the scope of the present example embodiment.

Note that, in a case where the series of processes in the information processing method illustrated in FIG. 5 are carried out by the above information processing system in place of the information processing apparatus 4, an execution subject of steps S41 through S43 is the foregoing feature quantity extraction apparatus, and an execution subject of step S43 is the foregoing estimation apparatus. Of course, it is possible to employ a configuration in which the foregoing estimation apparatus carries out the processes of steps S41 through S44.

In the following descriptions, an example will be described in which a stress level of a subject in one month is estimated while using, as biological signals, heart rate data and perspiration data which have been measured by the wearable terminal 7 for the one month. Note, however, that the measurement period can be less than one month or can be longer than one month. Further, the “feature quantity” in FIG. 5 is the above described estimating feature quantity and, in the description of FIG. 5, each of feature quantities is simply referred to as a feature quantity.

In step S41, the biological signal acquisition section 401 acquires biological signals. As described above, biological signals acquired here are heart rate data and perspiration data of the subject which have been measured in one month by the wearable terminal 7. Then, the biological signal acquisition section 401 causes the storage section 41 to store the acquired biological signals as biological signals 411.

In step S42, the identification section 404 identifies a time zone of interest. The process which is carried out in step S42 for identifying a time zone of interest is similar to the foregoing identification process which is carried out in step S32 in the training phase. That is, in the present example embodiment, predetermined time zones (e.g., from 7:00 to 13:00) before and after a time in the morning at which a predetermined index value of a biological signal reaches a peak based on a circadian rhythm are identified as the time zone of interest.

In step S43, the extraction section 405 extracts feature quantities from biological signals measured in the time zone of interest which has been identified in step S42, among the biological signals 411 which have been stored in step S41. The extracted feature quantities are stored in the storage section 41 as feature quantity data 414.

In step S44, the estimation section 409 estimates the stress level of the subject. Specifically, the estimation section 409 inputs, into the estimation model 416, the feature quantity which has been extracted in step S43. This estimation model 416 is the estimation model generated in step S36 in FIG. 4. Then, the estimation section 409 causes the storage section 41 to store an output value of the estimation model 416 as estimation result data 417. Note that the estimation section 409 may cause the output section 43 to output the estimated stress level. Thus, the stress level estimation method ends.

As described above, the information processing apparatus 4 in accordance with the present example embodiment employs the configuration of including the identification section 404 and the extraction section 405 described above. In particular, the identification section 404 is configured to identify, as a time zone of interest, predetermined time zones before and after a time in the morning at which a predetermined index value of a biological signal reaches a peak based on a circadian rhythm.

According to the above configuration, a feature quantity is extracted from a biological signal which has been acquired in a predetermined time zone (e.g., in the morning) in which a predetermined index value of the biological signal is relatively high in one day, including a time (e.g., at around 10:00) at which the predetermined index value of the biological signal (e.g., a heart rate) reaches a peak in one day. The inventors of the present invention have conceived that a chronic stress tendency is notably shown in biological signals in a time zone in which a predetermined index value of a biological signal (e.g., a heart rate, an amount of perspiration, or the like) is relatively high. Furthermore, it is known that a predetermined index value of a biological signal reaches, based on a circadian rhythm, a peak at around a time in the morning (e.g., 10:00 a.m.) and tends to be high in predetermined time zones before and after that time in the morning.

Therefore, a feature quantity is extracted from a biological signal in predetermined time zones before and after a time in the morning at which a predetermined index value of a biological signal reaches a peak based on a circadian rhythm. Thus, it is possible to bring about an example advantage of extracting an adequate feature quantity which is used in machine learning of a stress level estimation model or in estimation of a stress level using the estimation model. Moreover, it is possible to efficiently construct an estimation model that achieves high estimation accuracy of chronic stress, and to carry out estimation of chronic stress with higher accuracy.

As described above, the training data generation method in accordance with the present example embodiment employs the configuration of including step S35 of generating training data for use in machine learning by associating, as correct answer data, a stress level of a subject with one or more feature quantities which have been extracted by the feature quantity extraction method including steps S32 and S33. Therefore, according to the training data generation method in accordance with the present example embodiment, it is possible to bring about an example advantage of generating training data capable of efficiently constructing an estimation model that achieves high estimation accuracy of chronic stress.

As described above, the estimation model generation method in accordance with the present example embodiment employs the configuration of including step S36 of generating an estimation model by machine learning using training data which has been generated by the training data generation method including step S35. Therefore, according to the estimation model generation method in accordance with the present example embodiment, it is possible to bring about an example advantage of generating an estimation model that achieves high estimation accuracy of chronic stress.

As described above, the stress level estimation method in accordance with the present example embodiment employs the configuration of including step S44 of estimating a stress level of a subject using an estimation model which has been generated by the estimation model generation method including step S36. Therefore, according to the estimation method in accordance with the present example embodiment, it is possible to bring about an example advantage of accurately estimating a stress level related to chronic stress.

### Third Example Embodiment

The following description will discuss a third example embodiment of the present invention in detail with reference to the drawings. The same reference numerals are given to constituent elements which have functions identical with those described in the foregoing example embodiments, and descriptions as to such constituent elements are not repeated.

In the present example embodiment, a feature quantity is extracted for each attribute of a subject, training data is generated for each attribute, and an estimation model is generated for each attribute. Then, in estimating a stress level of a subject, a stress level of the subject is estimated using an estimation model corresponding to an attribute of the subject. The attribute of the subject can be, for example, gender.

In the present example embodiment, for example, a time zone of interest in which a chronic stress tendency is notably shown in biological signals is identified based on an eating time zone.

Generally, a predetermined index value of a biological signal (e.g., a heart rate, an amount of perspiration, or the like) tends to increase with eating. Furthermore, an amount of food intake increases when a male is in a chronic stress tendency. Therefore, the inventors of the present invention have paid attention to the fact that a chronic stress tendency is notably shown in a predetermined index of a biological signal in an eating time zone. In view of this, in the present example embodiment, for example, a standard lunch time zone is identified for a male subject as a time zone of interest in which a chronic stress tendency is notably shown in biological signals.

Further, the inventors of the present invention have inferred that a predetermined index value of a biological signal of a female tends to decrease in an eating time zone, and that manifestation of a chronic stress tendency is blunted. In view of this, in the present example embodiment, for example, a time zone other than the standard lunch time zone is identified for a female subject as a time zone of interest. For example, an ultimate time zone of interest may be identified by excluding a standard lunch time zone from a time zone of interest which has been identified based on the first or second example embodiment.

Note that the time zone of interest is identified based on the lunch time zone because, among all time zones, the lunch time zone does not vary widely among individuals. Note that the lunch time zone is more suitable as a time zone of interest because there is even less variation if limited to working days.

### <Configuration of Information Processing Apparatus>

In the present example embodiment, the identification section 404 is configured to identify a time zone of interest based on a predetermined lunch time zone. For example, a questionnaire may be answered in advance for a lunch time zone from a subject, and a most standard time zone (e.g., from 12:00 to 13:00) may be determined as the lunch time zone. Specifically, the identification section 404 identifies, as a time zone of interest for a male subject, the above standard lunch time zone in one day. Moreover, the identification section 404 identifies, as a time zone of interest for a female subject, a time zone other than the above standard lunch time zone in one day. For example, in a case where a time zone of interest is identified in the manner described in the first example embodiment or the second example embodiment and then the time zone of interest includes the above standard lunch time zone, the identification section 404 may extrude the standard lunch time zone from that time zone of interest.

In the present example embodiment, the extraction section 405 extracts a feature quantity for each gender based on the time zone of interest which has been identified by the identification section 404 for each gender.

Specifically, the extraction section 405 extracts, for a male subject, a feature quantity from a biological signal acquired in the lunch time zone which has been identified. Hereinafter, the feature quantity for a male which has been extracted from the biological signal acquired in the lunch time zone is referred to as a first feature quantity. Among the first feature quantities, a feature quantity used for generation of training data 415 is referred to as a first training feature quantity. Among the first feature quantities, a feature quantity used for estimation of a stress level is referred to as a first estimating feature quantity.

Specifically, the extraction section 405 extracts, for a female subject, a feature quantity from a biological signal acquired in the time zone other than the lunch time zone. Hereinafter, the feature quantity for a female which has been extracted from a biological signal acquired in the time zone other than the lunch time zone is referred to as a second feature quantity. Among the second feature quantities, a feature quantity used for generation of training data 415 is referred to as a second training feature quantity. Among the second feature quantities, a feature quantity used for estimation of a stress level is referred to as a second estimating feature quantity.

That is, in the present example embodiment, the feature quantity data 414 includes a first training feature quantity, a first estimating feature quantity, a second training feature quantity, and a second estimating feature quantity.

In the present example embodiment, the training data generation section 407 generates training data by associating, as correct answer data, a stress level indicated in the stress level data 413 of a male subject with a combination of one or more first training feature quantities. The first training feature quantity described above is extracted from a biological signal of a male subject by the extraction section 405. The training data generated as described above is used to construct an estimation model for males. Hereinafter, training data for constructing an estimation model for males is referred to as first training data.

Moreover, the training data generation section 407 generates training data by associating, as correct answer data, a stress level indicated in the stress level data 413 of a female subject with a combination of one or more second training feature quantities. The second training feature quantity described above is extracted from a biological signal of a female subject by the extraction section 405. The training data generated as described above is used to construct an estimation model for females. Hereinafter, training data for constructing an estimation model for females is referred to as second training data.

That is, in the present example embodiment, the training data 415 includes first training data and second training data.

In the present example embodiment, the training process section 408 generates, by training using the first training data, an estimation model for which the one or more first training feature quantities extracted by the extraction section 405 are used as explanatory variables and from which the stress level of the male subject is obtained as an objective variable. Hereinafter, the above estimation model for estimating a stress level of a male subject is referred to as a first estimation model.

Moreover, the training process section 408 generates, by training using the second training data, an estimation model for which the one or more second training feature quantities extracted by the extraction section 405 are used as explanatory variables and from which the stress level of the female subject is obtained as an objective variable. Hereinafter, the above estimation model for estimating a stress level of a female subject is referred to as a second estimation model.

That is, in the present example embodiment, the estimation model 416 includes a first estimation model and a second estimation model.

The estimation section 409 estimates a stress level of a subject using the first estimation model when the subject to be estimated is a male, and using the second estimation model when the subject to be estimated is a female.

### <Flow of Information Processing Method in Training Phase>

A flow of an information processing method in a training phase, which is carried out by the information processing apparatus 4 in accordance with the third example embodiment of the present invention, is described with reference to FIG. 4. The information processing method of the third example embodiment differs from the information processing method of the second example embodiment as follows.

In step S32, the identification section 404 identifies, as a time zone of interest, a standard lunch time zone in one day when the biological signal acquired in step S31 is a biological signal of a male subject. When the biological signal acquired in step S31 is a biological signal of a female subject, the identification section 404 identifies, as a time zone of interest, a time zone other than a standard lunch time zone in one day.

In step S33, the extraction section 405 extracts a first training feature quantity from a biological signal acquired in the above lunch time zone which has been identified, when the biological signal acquired in step S31 is a biological signal of a male subject. When the biological signal acquired in step S31 is a biological signal of a female subject, the extraction section 405 extracts a second training feature quantity from a biological signal acquired in the above time zone other than the lunch time zone.

In step S35, the training data generation section 407 generates first training data when the biological signal acquired in step S31 is a biological signal of a male subject. The first training data is generated by associating, as correct answer data, a stress level which has been calculated in step S34 with a combination of first training feature quantities which have been extracted in step S33. The training data generation section 407 generates second training data when the biological signal acquired in step S31 is a biological signal of a female subject. The second training data is generated by associating, as correct answer data, a stress level which has been calculated in step S34 with a combination of second training feature quantities which have been extracted in step S33.

In a case where the biological signal acquired in step S31 is a biological signal of a male subject, the training process section 408 generates, in step S36, a first estimation model for estimating a stress level of a male subject, by machine learning using the first training data generated in step S35. In a case where the biological signal acquired in step S31 is a biological signal of a female subject, the training process section 408 generates a second estimation model for estimating a stress level of a female subject, by machine learning using the second training data generated in step S35.

### <Flow of Information Processing Method in Inference Phase>

A flow of an information processing method in an inference phase, which is carried out by the information processing apparatus 4 in accordance with the third example embodiment of the present invention, is described with reference to FIG. 5. The information processing method of the third example embodiment differs from the information processing method of the second example embodiment as follows.

In step S42, the identification section 404 identifies, as a time zone of interest, a standard lunch time zone in one day when the biological signal acquired in step S41 is a biological signal of a male subject. When the biological signal acquired in step S41 is a biological signal of a female subject, the identification section 404 identifies, as a time zone of interest, a time zone other than a standard lunch time zone in one day.

In step S43, the extraction section 405 extracts a first estimating feature quantity from a biological signal acquired in the above lunch time zone which has been identified, when the biological signal acquired in step S41 is a biological signal of a male subject. When the biological signal acquired in step S41 is a biological signal of a female subject, the extraction section 405 extracts a second estimating feature quantity from a biological signal acquired in the above time zone other than the lunch time zone.

In a case where the biological signal acquired in step S41 is a biological signal of a male subject, the estimation section 409 inputs, in step S44, the first estimating feature quantity extracted in step S43 into the first estimation model generated in step S36. The estimation section 409 causes the storage section 41 to store an output value of the first estimation model as estimation result data 417 of the foregoing male subject. In a case where the biological signal acquired in step S41 is a biological signal of a female subject, the estimation section 409 inputs the second estimating feature quantity extracted in step S43 into the second estimation model generated in step S36. The estimation section 409 causes the storage section 41 to store an output value of the second estimation model as estimation result data 417 of the foregoing female subject.

As described above, the information processing apparatus 4 in accordance with the present example embodiment employs the configuration of including the identification section 404 and the extraction section 405 described above. In particular, for a male subject, the identification section 404 is configured to identify, as a time zone of interest, a standard lunch time zone of the subject in one day. Moreover, in particular, for a male subject, the extraction section 405 is configured to extract a feature quantity from a biological signal acquired in the lunch time zone which has been identified.

According to the above configuration, a feature quantity is extracted from a biological signal of a male subject which has been acquired in a lunch time zone that is an eating time zone in which, for males, a predetermined index value of a biological signal (e.g., a heart rate, an amount of perspiration, or the like) tends to be relatively high in one day, and that particularly does not vary widely among individuals. The eating time zone is considered to be a time zone in which a chronic stress tendency is notable in biological signals of males. Therefore, by narrowing down biological signals from which feature quantities are to be extracted into biological signals in the lunch time zone, it is possible to efficiently construct an estimation model that achieves high estimation accuracy of chronic stress of a male, and to carry out estimation of chronic stress of a male with higher accuracy.

As described above, the information processing apparatus 4 in accordance with the present example embodiment employs the configuration of including the identification section 404 and the extraction section 405 described above. In particular, for a female subject, the identification section 404 is configured to identify, as a time zone of interest, a time zone other than a standard lunch time zone of the subject in one day. Moreover, in particular, for a female subject, the extraction section 405 is configured to extract a feature quantity from a biological signal acquired in the time zone other than the lunch time zone.

According to the above configuration, a feature quantity is extracted while excluding biological signals in a lunch time zone that is an eating time zone in which, for females, a predetermined index value of a biological signal (e.g., a heart rate, an amount of perspiration, or the like) tends to be relatively low in one day, and that particularly does not vary widely among individuals. When a female is in a chronic stress tendency, an amount of food intake decreases. Therefore, it can be inferred that biological signals associated with eating are blunted in an eating time zone. In view of this, at least the lunch time zone is excluded from the time zone of interest, and biological signals from which feature quantities are to be extracted are narrowed down. By doing so, it is possible to efficiently construct an estimation model having improved estimation accuracy of chronic stress of a female, and to improve estimation accuracy of chronic stress of a female.

### Fourth Example Embodiment

The following description will discuss a fourth example embodiment of the present invention in detail with reference to the drawings. The same reference numerals are given to constituent elements which have functions identical with those described in the foregoing example embodiments, and descriptions as to such constituent elements are not repeated.

In the present example embodiment, a feature quantity is extracted for each attribute of a subject, training data is generated for each attribute, and an estimation model is generated for each attribute. Then, in estimating a stress level of a subject, a stress level of the subject is estimated using an estimation model corresponding to an attribute of the subject. The attribute of the subject can be, for example, gender.

In the present example embodiment, for example, a time zone of interest in which a chronic stress tendency is notably shown in biological signals is identified based on a time zone in which a subject is exposed to an acute stress stimulus. Hereinafter, the time zone in which a subject is exposed to an acute stress stimulus is referred to as a stress occurring time zone.

The inventors of the present invention have inferred that a predetermined index value of a biological signal (e.g., a heart rate, an amount of perspiration, or the like) of a female tends to be high in an acute stress occurring time zone, and that a chronic stress tendency can be notably shown. In view of this, in the present example embodiment, for example, the above stress occurring time zone is identified for a female subject as a time zone of interest in which a chronic stress tendency is notably shown in biological signals.

Further, the inventors of the present invention have paid attention to the fact that manifestation of a predetermined index value of a biological signal of a male who is in a chronic stress tendency tends to be blunted in an acute stress occurring time zone, and therefore, manifestation of a chronic stress tendency that can be estimated by biological signals is blunted. In view of this, in the present example embodiment, for example, a time zone other than the above stress occurring time zone is identified for a male subject as a time zone of interest. For example, an ultimate time zone of interest may be identified by excluding a stress occurring time zone from a time zone of interest which has been identified based on the configuration of at least any of the first through third example embodiments.

### <Configuration of Information Processing Apparatus>

In the present example embodiment, as illustrated in FIG. 3, the control section 40 includes a determination section 406. The determination section 406 determines, based on biological signals, whether or not the subject is in a state of being exposed to an acute stress stimulus. For example, the determination section 406 may detect a stress occurring time zone by analyzing a biological signal as follows.

For example, the determination section 406 determines, using at least one of a heart rate indicated by heart rate data and an amount of perspiration indicated by perspiration data which have been obtained from a subject, whether or not the subject is in a state of being exposed to an acute stress stimulus.

For example, in a case where the heart rate data is data obtained by arranging, in time series, heart rates which have been measured in respective unit times, the determination section 406 may compare the heart rate with a predetermined threshold for each unit time to determine whether or not there is exposure to an acute stress stimulus. For example, the determination section 406 may determine that “there is exposure to an acute stress stimulus” at a time point at which the heart rate is equal to or greater than the predetermined threshold. The determination section 406 may detect, as a stress occurring time zone, a set of time points at which it has been determined that “there is exposure to an acute stress stimulus”, and output the stress occurring time zone to the identification section 404.

In another example, the determination section 406 may detect, among the heart rate data, a time point SS at which a heart rate sharply rises, i.e., at which an increase in heart rate per unit time reaches or exceeds a predetermined threshold, and a time point EE at which the heart rate that has thus risen begins to decrease. Then, the determination section 406 may output, to the identification section 404, a time zone from the time point SS to the time point EE as a stress occurring time zone.

The determination section 406 may determine, using a plurality of types of biological signals, whether or not there is an acute stress stimulus, and detect a stress occurring time zone. For example, a time point at which an increase in heart rate and an increase in amount of perspiration have been simultaneously observed may be identified as a start time point of a stress occurring time zone, and a time point at which at least one of the heart rate and the amount of perspiration has decreased to a normal level may be identified as an end time point of the stress occurring time zone. For example, the determination section 406 may detect, as a stress occurring time zone, a time period in which a pattern of variation of measured biological signals in a predetermined time period falls under a pattern peculiar to a state of being exposed to an acute stress stimulus.

The identification section 404 identifies, as a time zone of interest, a stress occurring time zone which has been determined by the determination section 406 for a female subject.

Moreover, the identification section 404 identifies, as a time zone of interest, a time zone other than the stress occurring time zone which has been determined by the determination section 406 for a male subject. For example, the identification section 404 may identify an ultimate time zone of interest by excluding the above stress occurring time zone from a time zone of interest which has been identified by the configuration of at least any of the first through third example embodiments.

The extraction section 405 extracts feature quantities separately for attributes, i.e., separately according to gender, based on the time zone of interest which has been identified, as with the third example embodiment. The feature quantity data 414 can include a first training feature quantity and a first estimating feature quantity for males and a second training feature quantity and a second estimating feature quantity for females.

The training data generation section 407 generates training data separately according to gender, as with the third example embodiment. The training data 415 can include first training data for males and second training data for females.

The training process section 408 generates estimation models separately according to gender, as with the third example embodiment. The estimation model 416 can include a first estimation model for males and a second estimation model for females.

The estimation section 409 estimates stress levels separately according to gender, as with the third example embodiment.

### <Flow of Information Processing Method in Training Phase>

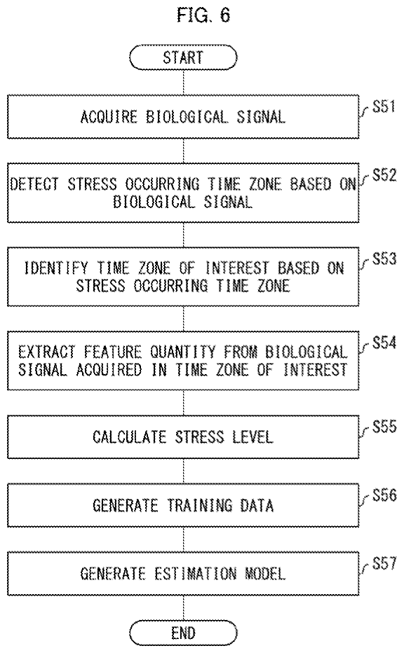

FIG. 6 is a flowchart illustrating a flow of an information processing method in a training phase, which is carried out by the information processing apparatus 4 in accordance with the fourth example embodiment of the present invention. The information processing method illustrated in FIG. 6 includes the feature quantity extraction method, the training data generation method, and the estimation model generation method in accordance with example aspects of the present invention, as with the second and third example embodiments. In the present example embodiment, steps S52 through S54 are processes for realizing the feature quantity extraction method, step S56 is a process for realizing the training data generation method, and step S57 is a process for realizing the estimation model generation method.

The processes describe above can also be realized by a program. That is, a feature quantity extraction program that causes a computer to carry out the processes of steps S52 through S54 is also encompassed in the scope of the present example embodiment. Similarly, a training data generation program that causes a computer to carry out the process in step S56 of generating training data using the feature quantity extracted in step S54 is also encompassed in the scope of the present example embodiment. Moreover, an estimation model generation program that causes a computer to carry out the process in step S57 of generating an estimation model using the training data generated in step S56 is also encompassed in the scope of the present example embodiment.

Note that a series of processes of the information processing method illustrated in FIG. 6 can be carried out by the above information processing system in place of the information processing apparatus 4. In this case, an execution subject of steps S51 through S54 is the foregoing feature quantity extraction apparatus, an execution subject of steps S55 and S56 is the foregoing training data generation apparatus, and an execution subject of step S57 is the foregoing estimation model generation apparatus.

In the following descriptions, an example will be described in which an estimation model is generated using, as biological signals, heart rate data and perspiration data of a subject which have been measured by the wearable terminal 7, as with the second and third example embodiments. In the information processing method of the fourth example embodiment described below, features in common with the foregoing information processing methods are described as “as with the . . . example embodiment” or “as with step S . . . ”, and thus the same descriptions will not be repeated.

In step S51, the biological signal acquisition section 401 acquires biological signals, as with step S31.

In step S52, the determination section 406 determines, based on a biological signal acquired in step S51, whether or not the subject is in a state of being exposed to an acute stress stimulus. For example, the determination section 406 may detect a stress occurring time zone by employing some specific determination methods described above.

In step S53, the identification section 404 identifies, as a time zone of interest, a time zone other than a stress occurring time zone, when the biological signal acquired in step S51 is a biological signal of a male subject. The identification section 404 identifies, as a time zone of interest, a stress occurring time zone when the biological signal acquired in S51 is a biological signal of a female subject.

In step S54, the extraction section 405 extracts a first training feature quantity from a biological signal acquired in a time zone other than a stress occurring time zone, when the biological signal acquired in step S51 is a biological signal of a male subject. The extraction section 405 extracts a second training feature quantity from a biological signal acquired in a stress occurring time zone, when the biological signal acquired in step S51 is a biological signal of a female subject.

In step S55, the stress level calculation section 403 calculates a stress level of the subject, as with step S34.

In step S56, the training data generation section 407 generates first training data when the biological signal acquired in step S51 is a biological signal of a male subject. The first training data is generated by associating, as correct answer data, a stress level which has been calculated in step S55 with a combination of first training feature quantities which have been extracted in step S54. The training data generation section 407 generates second training data when the biological signal acquired in step S51 is a biological signal of a female subject. The second training data is generated by associating, as correct answer data, a stress level which has been calculated in step S55 with a combination of second training feature quantities which have been extracted in step S54.

In a case where the biological signal acquired in step S51 is a biological signal of a male subject, the training process section 408 generates, in step S57, a first estimation model for estimating a stress level of a male subject, by machine learning using the first training data generated in step S56. In a case where the biological signal acquired in step S51 is a biological signal of a female subject, the training process section 408 generates a second estimation model for estimating a stress level of a female subject, by machine learning using the second training data generated in step S56.

### <Flow of Information Processing Method in Inference Phase>

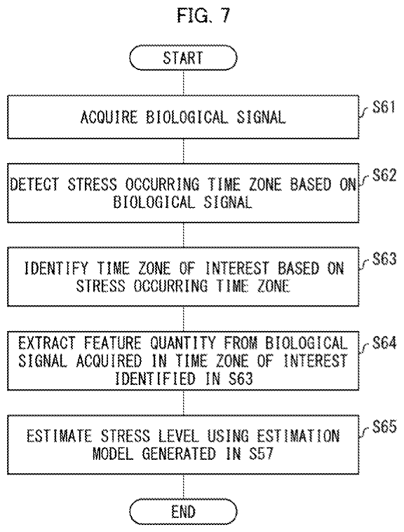

FIG. 7 is a flowchart illustrating a flow of an information processing method in an inference phase, which is carried out by the information processing apparatus 4 in accordance with the fourth example embodiment of the present invention. The information processing method illustrated in FIG. 7 includes, for example, the feature quantity extraction method and the stress level estimation method in accordance with example aspects of the present invention. In the present example embodiment, steps S62 through S64 are processes for realizing the feature quantity extraction method, and step S65 is a process for realizing the stress level estimation method.

The processes describe above can also be realized by a program. That is, a feature quantity extraction program that causes a computer to carry out the processes of steps S62 through S64 is also encompassed in the scope of the present example embodiment. Moreover, a stress level estimation program that causes a computer to carry out the process in step S65 of estimating a stress level using the estimation model generated in step S57 is also encompassed in the scope of the present example embodiment.

Note that, in a case where the series of processes in the information processing method illustrated in FIG. 7 are carried out by the above information processing system in place of the information processing apparatus 4, an execution subject of steps S61 through S64 is the foregoing feature quantity extraction apparatus, and an execution subject of step S65 is the foregoing estimation apparatus.

In the following descriptions, an example will be described in which a stress level of a subject in one month is estimated while using, as biological signals, heart rate data and perspiration data which have been measured for one month by the wearable terminal 7, as with the second and third example embodiments. In the information processing method of the fourth example embodiment described below, features in common with the foregoing information processing methods are described as “as with the . . . example embodiment” or “as with step S . . . ”, and thus the same descriptions will not be repeated.

In step S61, the biological signal acquisition section 401 acquires biological signals, as with step S41.

In step S62, the determination section 406 determines, as with step S52, whether or not the subject is in a state of being exposed to an acute stress stimulus. For example, the determination section 406 may detect a stress occurring time zone by employing some specific determination methods described above.

In step S63, the identification section 404 identifies, as a time zone of interest, a time zone other than a stress occurring time zone, when the biological signal acquired in step S61 is a biological signal of a male subject. The identification section 404 identifies, as a time zone of interest, a stress occurring time zone when the biological signal acquired in step S61 is a biological signal of a female subject.

In step S64, the extraction section 405 extracts a first estimating feature quantity from a biological signal acquired in a time zone other than a stress occurring time zone, when the biological signal acquired in step S61 is a biological signal of a male subject. The extraction section 405 extracts a second estimating feature quantity from a biological signal acquired in a stress occurring time zone, when the biological signal acquired in step S61 is a biological signal of a female subject.

In a case where the biological signal acquired in step S61 is a biological signal of a male subject, the estimation section 409 inputs, in step S65, the first estimating feature quantity extracted in step S64 into the first estimation model generated in step S57. The estimation section 409 causes the storage section 41 to store an output value of the first estimation model as estimation result data 417 of the foregoing male subject. In a case where the biological signal acquired in step S61 is a biological signal of a female subject, the estimation section 409 inputs the second estimating feature quantity extracted in step S64 into the second estimation model generated in step S57. The estimation section 409 causes the storage section 41 to store an output value of the second estimation model as estimation result data 417 of the foregoing female subject.

As described above, the information processing apparatus 4 in accordance with the present example embodiment employs the configuration of including the determination section 406, the identification section 404, and the extraction section 405 described above. The determination section 406 is configured to determine, based on a biological signal, whether or not the subject is in a state of being exposed to an acute stress stimulus. The identification section 404 is configured to identify, as a time zone of interest for a female subject, a stress occurring time zone in which the subject has been determined to be in a state of being exposed to an acute stress stimulus. The extraction section 405 is configured to extract, for a female subject, a feature quantity from a biological signal acquired in the stress occurring time zone which has been identified.

It is inferred that, in an acute stress occurring time zone, a chronic stress tendency is notable in biological signals for a female. According to the above configuration, among biological signals of a female subject, a feature quantity is extracted from a biological signal which has been acquired in the stress occurring time zone. Therefore, by narrowing down biological signals from which feature quantities are to be extracted into biological signals in the stress occurring time zone, it is possible to efficiently construct an estimation model that achieves high estimation accuracy of chronic stress of a female, and to carry out estimation of chronic stress of a female with higher accuracy.

As described above, the information processing apparatus 4 in accordance with the present example embodiment employs the configuration of including the determination section 406, the identification section 404, and the extraction section 405 described above. The determination section 406 is configured to determine, based on a biological signal, whether or not the subject is in a state of being exposed to an acute stress stimulus. The identification section 404 is configured to identify, as a time zone of interest for a male subject, a time zone other than a stress occurring time zone in which the subject has been determined to be in a state of being exposed to an acute stress stimulus. The extraction section 405 is configured to extract, for a male subject, a feature quantity from a biological signal acquired in the time zone other than the stress occurring time zone.

In the acute stress occurring time zone, it is considered that, for a male, manifestation of a chronic stress tendency in biological signals is blunted. According to the above configuration, among biological signals of a male subject, a feature quantity is extracted from a biological signal which has been acquired in the time zone other than the stress occurring time zone. Thus, it is possible to efficiently construct an estimation model that achieves high estimation accuracy of chronic stress of a male, and to carry out estimation of chronic stress of a male with higher accuracy.

### Fifth Example Embodiment

The following description will discuss a fifth example embodiment of the present invention in detail with reference to the drawings. The same reference numerals are given to constituent elements which have functions identical with those described in the foregoing example embodiments, and descriptions as to such constituent elements are not repeated.

In the present example embodiment, an attribute of a subject who has provided acquired biological signals is determined, and information processing is carried out differently for each of the determined attributes.

### <Configuration of Information Processing Apparatus>

In the present example embodiment, the biological signal acquisition section 401 illustrated in FIG. 3 acquires, together with a biological signal, attribute information indicating an attribute of a subject who provides the biological signal. In the present example embodiment, the attribute of the subject is, for example, gender. Therefore, in the present example embodiment, the attribute information is information indicating a gender of the subject.

### <Flow of Information Processing Method in Training Phase>

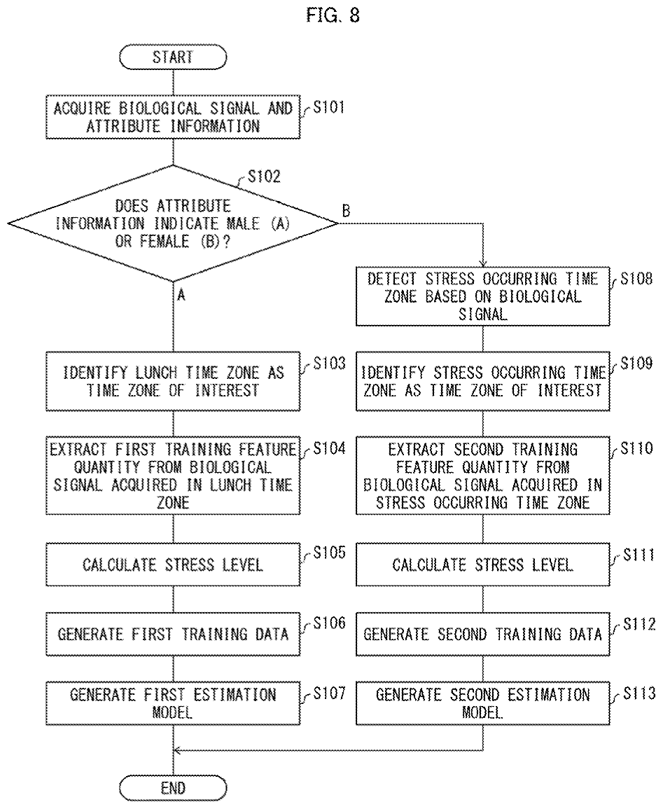

FIG. 8 is a flowchart illustrating a flow of an information processing method in a training phase, which is carried out by the information processing apparatus 4 in accordance with the fifth example embodiment of the present invention. In the following descriptions, an example will be described in which an estimation model is generated using, as biological signals, heart rate data and perspiration data of a subject which have been measured by the wearable terminal 7, as with the foregoing example embodiments. In the information processing method of the fifth example embodiment described below, features in common with the foregoing information processing methods are described as “as with the . . . example embodiment” or “as with step S . . . ”, and thus the same descriptions will not be repeated.

In step S101, the biological signal acquisition section 401 acquires a biological signal used for generation of an estimation model and attribute information of a subject who provides the biological signal. For example, the wearable terminal 7 may transmit attribute information of a wearer of the wearable terminal 7 which has been registered in advance to the information processing apparatus 4 together with a biological signal.

In step S102, the identification section 404 determines whether the attribute information acquired in step S101 indicates a male or a female. In a case where the attribute information indicates a male, the identification section 404 advances the process from A in step S102 to step S103. In a case where the attribute information indicates a female, the identification section 404 advances the process from B in step S102 to step S108.

In step S103, the identification section 404 identifies, as a time zone of interest, a predetermined standard lunch time zone among biological signals of a male subject, as with step S32 in the third example embodiment.

In step S104, the extraction section 405 extracts a first training feature quantity from a biological signal which has been acquired in the lunch time zone, as with step S33 in the third example embodiment.

In step S105, the stress level calculation section 403 calculates a stress level, as with step S34 in the foregoing example embodiments.

In step S106, the training data generation section 407 generates first training data by associating a stress level with the first training feature quantity, as with step S35 in the third example embodiment.

In step S107, the training process section 408 generates a first estimation model by machine learning using the first training data, as with step S36 in the third example embodiment.

In step S108, the determination section 406 determines, based on a biological signal, whether or not the subject is in a state of being exposed to an acute stress stimulus, as with step S52. For example, the determination section 406 may detect a stress occurring time zone.

In step S109, the identification section 404 identifies, as a time zone of interest, a stress occurring time zone, as with step S53.

In step S110, the extraction section 405 extracts a second training feature quantity from a biological signal which has been acquired in the stress occurring time zone, as with step S54.

In step S111, the stress level calculation section 403 calculates a stress level, as with steps S34 and S55 in the foregoing example embodiments.

In step S112, the training data generation section 407 generates second training data by associating a stress level with the second training feature quantity, as with step S56.

In step S113, the training process section 408 generates a second estimation model by machine learning using the second training data, as with step S57.

### <Flow of Information Processing Method in Inference Phase>

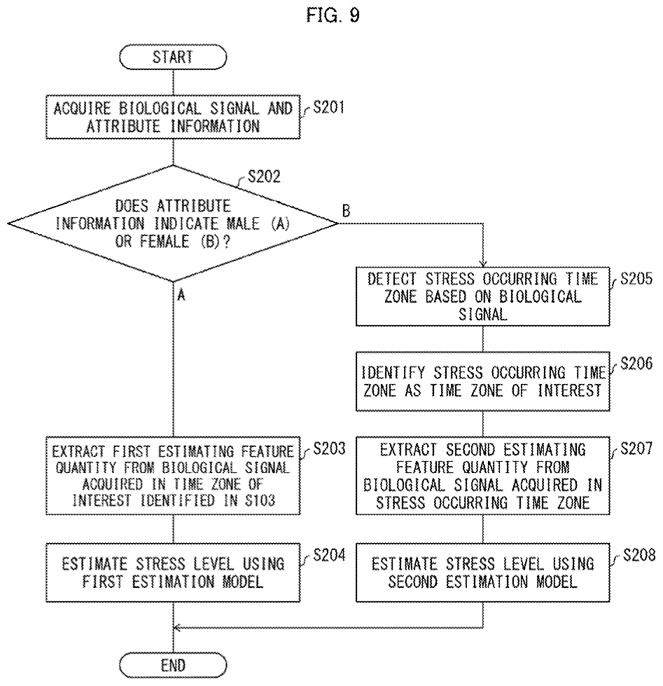

FIG. 9 is a flowchart illustrating a flow of an information processing method in an inference phase, which is carried out by the information processing apparatus 4 in accordance with the fifth example embodiment of the present invention. In the following descriptions, an example will be described in which a stress level of a subject in one month is estimated while using, as biological signals, heart rate data and perspiration data which have been measured for one month by the wearable terminal 7, as with the foregoing example embodiments. In the information processing method of the fifth example embodiment described below, features in common with the foregoing information processing methods are described as “as with the . . . example embodiment” or “as with step S . . . ”, and the same descriptions will not be repeated.

In step S201, the biological signal acquisition section 401 acquires a biological signal used for estimation of a stress level of a subject and attribute information of the subject who provides the biological signal. As with step S101, the attribute information may be transmitted from the wearable terminal 7 to the information processing apparatus 4 together with the biological signal.

In step S202, the identification section 404 determines a gender indicated by the attribute information, as with step S102. In a case where the attribute information indicates a male, the identification section 404 advances the process from A in step S202 to step S203. In a case where the attribute information indicates a female, the identification section 404 advances the process from B in step S202 to step S205.

In step S203, the extraction section 405 extracts a first estimating feature quantity from a biological signal acquired in the time zone of interest which has been identified in step S103, that is, a standard lunch time zone.

In step S204, the estimation section 409 estimates, with use of the first estimation model generated in step S107, a stress level of a male subject who is a provider of the biological signal acquired in step S201, as with step S65. Specifically, the first estimating feature quantity which has been extracted in step S203 is input into the first estimation model which has been generated in step S107. Then, the estimation section 409 causes the storage section 41 to store an output value of the first estimation model as estimation result data 417 of the foregoing male subject.

In step S205, the determination section 406 determines, based on biological signals, whether or not the subject is in a state of being exposed to an acute stress stimulus, as with step S108. For example, the determination section 406 may detect a stress occurring time zone.

In step S206, the identification section 404 identifies, as a time zone of interest, a stress occurring time zone, as with step S109.

In step S207, the extraction section 405 extracts a second estimating feature quantity from a biological signal which has been acquired in the stress occurring time zone, as with step S64.

In step S208, the estimation section 409 estimates, with use of the second estimation model generated in step S113, a stress level of a female subject who is a provider of the biological signal acquired in step S201, as with step S65. Specifically, the second estimating feature quantity which has been extracted in step S207 is input into the second estimation model which has been generated in step S113. Then, the estimation section 409 causes the storage section 41 to store an output value of the second estimation model as estimation result data 417 of the foregoing female subject.

According to each of the information processing methods in accordance with the present example embodiment, it is possible to extract a feature quantity for each of a male and a female, while focusing on a time zone in which a chronic stress tendency is notable. Specifically, for a male, a feature quantity is extracted from a biological signal which has been acquired in the lunch time zone. For a female, a feature quantity is extracted from a biological signal which has been acquired in the stress occurring time zone.

Thus, by more appropriately narrowing down a time zone in which a chronic stress tendency is notable according to gender, it is possible to efficiently construct, separately according to gender, an estimation model that achieves high estimation accuracy of chronic stress, and to carry out, separately according to gender, estimation of chronic stress with higher accuracy.

### [Variation]

In each of the foregoing example embodiments, an example has been described in which an attribute of a subject is gender. However, the attribute only needs to be an attribute related to a time zone in which a chronic stress tendency is notably shown in biological signals, and is not limited to gender. For example, a time zone of interest may be identified which corresponds to an attribute of a subject such as an age group or an occupation of the subject. Moreover, it is possible that a feature quantity is extracted, in accordance with an attribute (such as an age group or an occupation) of the subject, from a biological signal acquired in a time zone of interest which has been identified as described above, and an estimation model is constructed for each attribute such as an age group or an occupation of the subject using the feature quantity. Then, a feature quantity corresponding to the attribute such as an age group or an occupation of the subject is input into the estimation model which has been constructed as described above, and it is thus possible to estimate a stress level with high accuracy according to the attribute such as an age group or an occupation of the subject.

### [Software Implementation Example]

The functions of part of or all of the information processing apparatuses (1 and 4) can be realized by hardware such as an integrated circuit (IC chip) or can be alternatively realized by software.

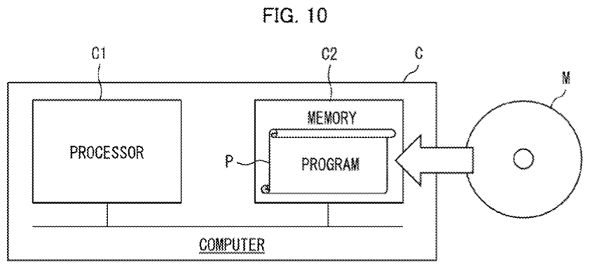

In the latter case, each of the foregoing information processing apparatuses is realized by, for example, a computer that executes instructions of a program that is software realizing the foregoing functions. FIG. 10 illustrates an example of such a computer (hereinafter, referred to as “computer C”). The computer C includes at least one processor C1 and at least one memory C2. The memory C2 stores a program P for causing the computer C to function as the foregoing information processing apparatuses. In the computer C, the processor C1 reads the program P from the memory C2 and executes the program P, so that the functions of the foregoing information processing apparatuses are realized.

Examples of the processor C1 can include a central processing unit (CPU), a graphic processing unit (GPU), a digital signal processor (DSP), a micro processing unit (MPU), a floating point number processing unit (FPU), a physics processing unit (PPU), a microcontroller, and a combination thereof. Examples of the memory C2 include a flash memory, a hard disk drive (HDD), a solid state drive (SSD), and a combination thereof.

Note that the computer C can further include a random access memory (RAM) in which the program P is loaded when the program P is executed and in which various kinds of data are temporarily stored. The computer C can further include a communication interface for carrying out transmission and reception of data with other apparatuses. The computer C can further include an input-output interface for connecting input-output apparatuses such as a keyboard, a mouse, a display and a printer.

The program P can be stored in a non-transitory tangible storage medium M which is readable by the computer C. Examples of such a storage medium M can include a tape, a disk, a card, a semiconductor memory, and a programmable logic circuit. The computer C can obtain the program P via the storage medium M. Alternatively, the program P can be transmitted via a transmission medium. Examples of such a transmission medium can include a communication network and a broadcast wave. The computer C can obtain the program P also via such a transmission medium.

### [Additional Remark 1]

The present invention is not limited to the foregoing example embodiments, but may be altered in various ways by a skilled person within the scope of the claims. For example, the present invention also encompasses, in its technical scope, any example embodiment derived by appropriately combining technical means disclosed in the foregoing example embodiments.

### [Additional Remark 2]

Some or all of the foregoing example embodiments can also be described as below. Note, however, that the present invention is not limited to the following supplementary notes.

### (Supplementary Note 1)

An information processing apparatus, including: an identification means of identifying, as a time zone of interest, a time zone in which a chronic stress tendency is notably shown in biological signals which have been acquired from a subject over a predetermined time period; and an extraction means of extracting one or more feature quantities from biological signals acquired in the time zone of interest which has been identified, the one or more feature quantities being used in machine learning of an estimation model for estimating a stress level or used in estimation of a stress level using the estimation model.

According to the above configuration, it is possible to bring about an example advantage of extracting an adequate feature quantity which is used in machine learning of a stress level estimation model or in estimation of a stress level using the estimation model.

### (Supplementary Note 2)

The information processing apparatus according to supplementary note 1, in which: the identification means identifies, as the time zone of interest, a time zone in which the biological signals show a notable behavior in one day; and the extraction means extracts a feature quantity based on a change in a biological signal at a start time or an end time of the time zone of interest.

According to the above configuration, an example advantage below is brought about when it is known in advance that an aspect of a change in a biological signal due to a transition of a subject to a particular state or the like is significantly correlated with chronic stress. That is, by extracting a feature quantity based on the above change, it is possible to bring about an example advantage of increasing estimation accuracy of chronic stress.

### (Supplementary Note 3)

The information processing apparatus according to supplementary note 1 or 2, in which: the identification means identifies, as the time zone of interest, predetermined time zones before and after a time in the morning at which a predetermined index value of a biological signal reaches a peak based on a circadian rhythm.

According to the above configuration, a feature quantity is extracted from a biological signal in predetermined time zones before and after a time in the morning at which the index value reaches a peak based on a circadian rhythm. Therefore, it is possible to bring about an example advantage of extracting an adequate feature quantity which is used in machine learning of a stress level estimation model or in estimation of a stress level using the estimation model.

### (Supplementary Note 4)

The information processing apparatus according to any one of supplementary notes 1 through 3, in which: the identification means identifies, as the time zone of interest for a male subject, a standard lunch time zone of the subject in one day; and the extraction means extracts, for the male subject, a feature quantity from a biological signal acquired in the lunch time zone which has been identified.

According to the above configuration, it is possible to narrow down biological signals from which feature quantities are to be extracted into biological signals in the lunch time zone. Consequently, it is possible to efficiently construct an estimation model that achieves high estimation accuracy of chronic stress of a male, and to carry out estimation of chronic stress of a male with higher accuracy.

### (Supplementary Note 5)

The information processing apparatus according to any one of supplementary notes 1 through 4, in which: the identification means identifies, as the time zone of interest for a female subject, a time zone other than a standard lunch time zone of the subject in one day; and the extraction means extracts, for the female subject, a feature quantity from a biological signal acquired in the time zone other than the lunch time zone.

According to the above configuration, it is possible to narrow down biological signals from which feature quantities are to be extracted, by excluding at least the lunch time zone from the time zone of interest. Consequently, it is possible to efficiently construct an estimation model in which estimation accuracy of chronic stress of a female is improved, and to improve estimation accuracy of chronic stress of a female.

### (Supplementary Note 6)

The information processing apparatus according to any one of supplementary notes 1 through 5, further including: a determination means of determining, based on the biological signals, whether or not the subject is in a state of being exposed to an acute stress stimulus, the identification means identifying, as the time zone of interest for a female subject, a stress occurring time zone in which the subject has been determined to be in a state of being exposed to an acute stress stimulus, and the extraction means extracting, for the female subject, a feature quantity from a biological signal acquired in the stress occurring time zone which has been identified.

According to the above configuration, by narrowing down biological signals from which feature quantities are to be extracted into biological signals in the stress occurring time zone, it is possible to efficiently construct an estimation model that achieves high estimation accuracy of chronic stress of a female, and to carry out estimation of chronic stress of a female with higher accuracy.

### (Supplementary Note 7)

The information processing apparatus according to any one of supplementary notes 1 through 6, further including: a determination means of determining, based on the biological signals, whether or not the subject is in a state of being exposed to an acute stress stimulus, the identification means identifying, as the time zone of interest for a male subject, a time zone other than a stress occurring time zone in which the subject has been determined to be in a state of being exposed to an acute stress stimulus, and the extraction means extracting, for the male subject, a feature quantity from a biological signal acquired in the time zone other than the stress occurring time zone.

According to the above configuration, feature quantities are extracted while narrowing down biological signals into biological signals which have been acquired in the time zone other than the stress occurring time zone. Thus, it is possible to efficiently construct an estimation model that achieves high estimation accuracy of chronic stress of a male, and to carry out estimation of chronic stress of a male with higher accuracy.

### (Supplementary Note 8)

A feature quantity extraction method, including: identifying, as a time zone of interest by at least one processor, a time zone in which a chronic stress tendency is notably shown in biological signals which have been acquired from a subject over a predetermined time period; and extracting, by the at least one processor, one or more feature quantities from biological signals acquired in the time zone of interest which has been identified, the one or more feature quantities being used in machine learning of an estimation model for estimating a stress level or used in estimation of a stress level using the estimation model.

According to the above method, it is possible to bring about an example advantage similar to that of the information processing apparatus according to supplementary note 1.

### (Supplementary Note 9)

The feature quantity extraction method according to supplementary note 8, in which: in the identifying, the at least one processor identifies, as the time zone of interest, a time zone in which the biological signals show a notable behavior in one day; and in the extracting, the at least one processor extracts a feature quantity based on a change in a biological signal at a start time or an end time of the time zone of interest.

According to the above method, an example advantage below is brought about when it is known in advance that an aspect of a change in a biological signal due to a transition of a subject to a particular state or the like is significantly correlated with chronic stress. That is, by extracting a feature quantity based on the above change, it is possible to bring about an example advantage of increasing estimation accuracy of chronic stress.

### (Supplementary Note 10)

A training data generation method, including: generating, by at least one processor, training data for use in machine learning by associating, as correct answer data, a stress level of a subject with one or more feature quantities which have been extracted by a feature quantity extraction method described in supplementary note 8 or 9.

According to the above method, it is possible to bring about an example advantage of generating training data capable of efficiently constructing an estimation model that achieves high estimation accuracy of chronic stress.

### (Supplementary Note 11)

An estimation model generation method, including: generating, by at least one processor, an estimation model by machine learning using training data which has been generated by a training data generation method described in supplementary note 10.

According to the above method, it is possible to bring about an example advantage of generating an estimation model that achieves high estimation accuracy of chronic stress.

### (Supplementary Note 12)

A stress level estimation method, including: estimating, by at least one processor, a stress level of a subject using an estimation model which has been generated by an estimation model generation method described in supplementary note 11.

According to the above method, it is possible to bring about an example advantage of accurately estimating a stress level related to chronic stress.

### (Supplementary Note 13)

A feature quantity extraction program for causing a computer to function as: an identification means of identifying, as a time zone of interest, a time zone in which a chronic stress tendency is notably shown in biological signals which have been acquired from a subject over a predetermined time period; and an extraction means of extracting one or more feature quantities from biological signals acquired in the time zone of interest which has been identified, the one or more feature quantities being used in machine learning of an estimation model for estimating a stress level or used in estimation of a stress level using the estimation model. According to the above configuration, it is possible to bring about an example advantage similar to that of the information processing apparatus according to supplementary note 1.

### (Supplementary Note 14)

An estimation apparatus, including: an identification means of identifying, as a time zone of interest, a time zone in which a chronic stress tendency is notably shown in biological signals which have been acquired from a subject over a predetermined time period; an extraction means of extracting one or more feature quantities (estimating feature quantity) from biological signals acquired in the time zone of interest which has been identified, the one or more feature quantities being used in estimation of a stress level using an estimation model for estimating a stress level; and an estimation means of estimating a stress level of the subject based on an output value which has been obtained by inputting, into the estimation model, the one or more feature quantities which have been extracted.

### (Supplementary Note 15)

A stress level estimation method, including: identifying, as a time zone of interest by at least one processor, a time zone in which a chronic stress tendency is notably shown in biological signals which have been acquired from a subject over a predetermined time period; extracting, by the at least one processor, one or more feature quantities (estimating feature quantity) from biological signals acquired in the time zone of interest which has been identified, the one or more feature quantities being used in estimation of a stress level using an estimation model for estimating a stress level; and estimating, by the at least one processor, a stress level of the subject based on an output value which has been obtained by inputting, into the estimation model, the one or more feature quantities which have been extracted.

### [Additional Remark 3]

Some or all of the foregoing example embodiments can further be expressed as follows.

An information processing apparatus, including at least one processor, the at least one processor carrying out: an identification process of identifying, as a time zone of interest, a time zone in which a chronic stress tendency is notably shown in biological signals which have been acquired from a subject over a predetermined time period; and an extraction process of extracting one or more feature quantities from biological signals acquired in the time zone of interest which has been identified, the one or more feature quantities being used in machine learning of an estimation model for estimating a stress level or used in estimation of a stress level using the estimation model.

Note that the information processing apparatus can further include a memory. The memory can store a program for causing the at least one processor to carry out the identification process and the extraction process. The program can be stored in a computer-readable non-transitory tangible storage medium.

### REFERENCE SIGNS LIST

1: Information processing apparatus
4: Information processing apparatus
7: Wearable terminal
11: Identification section (identification means)
12: Extraction section (extraction means)
404: Identification section (identification means)
405: Extraction section (extraction means)
406: Determination section (determination means)
409: Estimation section (estimation means)

What is claimed is:

1. An information processing apparatus, comprising at least one processor, the at least one processor carrying out:
an identification process of identifying, as a time zone of interest, a time zone in which a chronic stress tendency is notably shown in biological signals which have been acquired from a subject over a predetermined time period; and
an extraction process of extracting one or more feature quantities from biological signals acquired in the time zone of interest which has been identified, the one or more feature quantities being used in machine learning of an estimation model for estimating a stress level or used in estimation of a stress level using the estimation model.

2. The information processing apparatus according to claim 1, wherein:
in the identification process, the at least one processor identifies, as the time zone of interest, a time zone in which the biological signals show a notable behavior in one day; and
in the extraction process, the at least one processor extracts a feature quantity based on a change in a biological signal at a start time or an end time of the time zone of interest.

3. The information processing apparatus according to claim 1, wherein:
in the identification process, the at least one processor identifies, as the time zone of interest, predetermined time zones before and after a time in the morning at which a predetermined index value of a biological signal reaches a peak based on a circadian rhythm.

4. The information processing apparatus according to claim 1, wherein:
in the identification process, the at least one processor identifies, as the time zone of interest for a male subject, a standard lunch time zone of the subject in one day; and
in the extraction process, the at least one processor extracts, for the male subject, a feature quantity from a biological signal acquired in the lunch time zone which has been identified.

5. The information processing apparatus according to claim 1, wherein:
in the identification process, the at least one processor identifies, as the time zone of interest for a female subject, a time zone other than a standard lunch time zone of the subject in one day; and
in the extraction process, the at least one processor extracts, for the female subject, a feature quantity from a biological signal acquired in the time zone other than the lunch time zone.

6. The information processing apparatus according to claim 1, wherein:
the at least one processor further carries out a determination process of determining, based on the biological signals, whether or not the subject is in a state of being exposed to an acute stress stimulus;
in the identification process, the at least one processor identifies, as the time zone of interest for a female subject, a stress occurring time zone in which the subject has been determined to be in a state of being exposed to an acute stress stimulus; and
in the extraction process, the at least one processor extracts, for the female subject, a feature quantity from a biological signal acquired in the stress occurring time zone which has been identified.

7. The information processing apparatus according to claim 1, wherein:
the at least one processor further carries out a determination process of determining, based on the biological signals, whether or not the subject is in a state of being exposed to an acute stress stimulus;
in the identification process, the at least one processor identifies, as the time zone of interest for a male subject, a time zone other than a stress occurring time zone in which the subject has been determined to be in a state of being exposed to an acute stress stimulus; and
in the extraction process, the at least one processor extracts, for the male subject, a feature quantity from a biological signal acquired in the time zone other than the stress occurring time zone.

8. A feature quantity extraction method, comprising:
identifying, as a time zone of interest by at least one processor, a time zone in which a chronic stress tendency is notably shown in biological signals which have been acquired from a subject over a predetermined time period; and
extracting, by the at least one processor, one or more feature quantities from biological signals acquired in the time zone of interest which has been identified, the one or more feature quantities being used in machine learning of an estimation model for estimating a stress level or used in estimation of a stress level using the estimation model.

9. The feature quantity extraction method according to claim 8, wherein:
in the identifying, the at least one processor identifies, as the time zone of interest, a time zone in which the biological signals show a notable behavior in one day; and
in the extracting, the at least one processor extracts a feature quantity based on a change in a biological signal at a start time or an end time of the time zone of interest.

10. A training data generation method, comprising:
generating, by at least one processor, training data for use in machine learning by associating, as correct answer data, a stress level of a subject with one or more feature quantities which have been extracted by a feature quantity extraction method recited in claim 8.

11. An estimation model generation method, comprising:
generating, by at least one processor, an estimation model by machine learning using training data which has been generated by a training data generation method recited in claim 10.

12. A stress level estimation method, comprising:
estimating, by at least one processor, a stress level of a subject using an estimation model which has been generated by an estimation model generation method recited in claim 11.

13. A computer-readable non-transitory storage medium storing a program for causing a computer to carry out:
an identification process of identifying, as a time zone of interest, a time zone in which a chronic stress tendency is notably shown in biological signals which have been acquired from a subject over a predetermined time period; and
an extraction process of extracting one or more feature quantities from biological signals acquired in the time zone of interest which has been identified, the one or more feature quantities being used in machine learning of an estimation model for estimating a stress level or used in estimation of a stress level using the estimation model.
